(19) United States
(12) Patent Application Publication
(10) Pub. No.: US 2007/0128297 A1
(43) Pub. Date: Jun. 7, 2007

(54) Apparatus and method for treatment of nasal congestion

(76) Inventor: LeTrinh Hoang, Altadena, CA (US)

Correspondence Address: James W. Wiegand;Suite 700, 60 State Street, Boston, MA, 02109, US

(21) Appl. No.: 11/295,946

(22) Filed: Dec. 7, 2005

(51) Int. Cl. A61K 33/14; A61M 31/00

(52) U.S. Cl. 424/680; 604/500

(57) ABSTRACT

An apparatus for treatment of sinus problems includes a single-use container with hypertonic saline solution. The concentration of Sodium chloride within the saline solution may fall within the range of 0.9% to 3.0%. The single-use container may be an ampule, or resilient, flexible bottle, for example. A package containing a plurality of single-use containers allows for several single-use applications of the hypertonic saline solution.

### FIELD OF THE INVENTION

The invention relates to the treatment of nasal congestion and, in particular, to a method and apparatus for the convenient delivery of a solution to the sinuses for irrigation of the sinuses.

### BACKGROUND OF THE INVENTION

Millions of people are stricken with the symptoms of colds, nasal congestion, allergies and nasal congestion on a daily basis. Such ailments may be accompanied by labored breathing, fatigue, and severe headaches, and may also be associated with serious medical complications, such as loss of hearing. The symptoms are particularly acute in the case of infants; infants are obligate nasal breathers and cannot feed if they are congested. Various treatments for colds nasal congestion, allergies and sinusitis exist and are employed with varying degrees of success. Many treatments include substances that may have deleterious side effects, many of the available treatments are not directed toward juvenile sinusitis, nasal congestion, and colds, and many treatments are not packaged in a convenient form.

An apparatus and method for the treatment of colds, nasal congestion, sinusitis, and allergies, particularly in infants would therefore be highly desirable.

### SUMMARY

In accordance with the principles of the present invention, hypertonic saline may be employed for the irrigation of sinuses in the treatment of colds, chronic nasal congestion and allergies, particularly for infants from approximately one month to six years of age. In an illustrative embodiment, the saline may range from greater than 0.9% to 3.0% and a volume of approximately 1 ml to 60 ml may be employed to irrigate the affected sinuses. In operation, a patient's head is tilted back and the hypertonic saline is introduced into one, or both, of the patient's nostrils and allowed to flow into the sinuses. Isotonic saline may also be employed in accordance with the principles of the present invention.

In accordance with another aspect of the invention, a sterile package contains sufficient hypertonic saline for a single application (a range of approximately 1 ml to 60 ml). Single-use packages ensure that the contents remain sterile until use and provide for convenient delivery of the appropriate amount of solution. The package may take any one of many forms, including various configurations of ampule, syringe, or squeeze bottle, for example. Additionally, a kit including a plurality of single-use packages may be employed for a course of treatment. The single-use packages within such a kit may be of the same or differing volumes and may contain the same or different concentrations of hypertonic saline.

### BRIEF DESCRIPTION OF THE DRAWINGS

The above and further features, aspects, and advantages of the invention will be apparent to those skilled in the art from the following detailed description, taken together with the accompanying drawings in which:

FIG. 1 is a flow chart that depicts the basic steps involved in a treatment in accordance with the principles of the present invention;

FIG. 2 is a schematic diagram of a container for use in a treatment in accordance with the principles of the present invention;

FIG. 3 is a schematic diagram of another container for use in a treatment in accordance with the principles of the present invention;

FIG. 4 is a schematic diagram of another container for use in a treatment in accordance with the principles of the present invention;

FIG. 5 is a schematic diagram of another container for use in a treatment in accordance with the principles of the present invention; and

FIG. 6 is a conceptual block diagram of a kit that includes a plurality of single-use hypertonic saline solution containers in accordance with the principles of the present invention.

### DETAILED DESCRIPTION

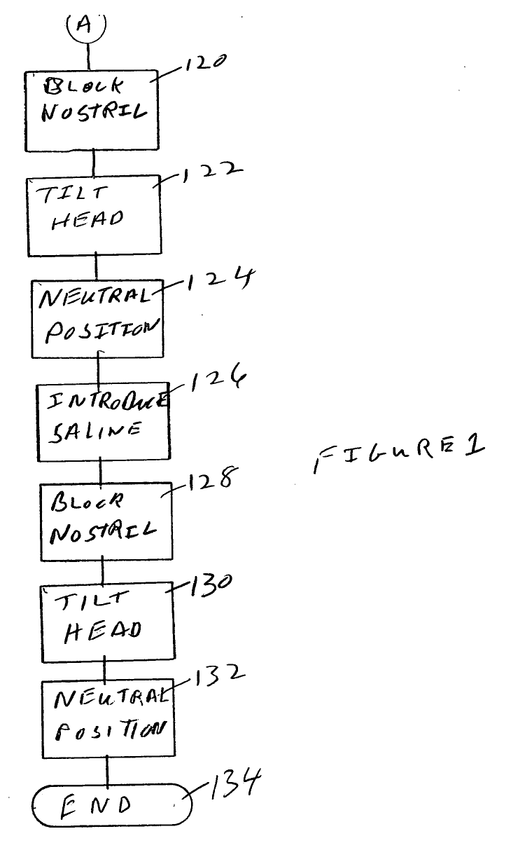

The flow chart of FIG. 1 illustrates a process in accordance with the principles of the present invention by which sinuses are irrigated using a hypertonic saline solution. The process begins in step 100 and proceeds from there to step 102, where hypertonic saline is introduced into one nostril of an affected individual. In an illustrative embodiment, the affected individual is a child between the ages of one month and six years. In this illustrative embodiment the child's head is tilted back to allow gravity to aid in the distribution of the saline.

After introducing the saline into the child's nostril, in step 104 a caregiver blocks that nostril in a manner that prevents the hypertonic saline solution from escaping through the opening in the nostril. Then, in step 106, the caregiver tilts the child's head in the direction opposite the nostril that received the hypertonic saline solution. That is, if, for example, the solution has been introduced to the child's left nostril, the caregiver tilts the child's head to the right. The solution is allowed to travel, on an interior path from the nostril into which the solution was introduced to the child's other nostril. In step 108, the caregiver returns the child's head to a neutral position (i.e., head tilted back, but tilted to neither the left or the right) and cleans the child's nostril, for example, using a suction bulb or syringe to suction fluid from the nostril that has had the hypertonic saline solution introduced.

The caregiver then, in step 110, again introduces hypertonic saline into and in step 112 blocks the child's nostril, as before. The caregiver then tilts the child's head in step 114 to the same side as the nostril into which they have just placed the hypertonic solution in order to irrigate the sinuses on that side of the child's head. In step 116 the caregiver again returns the child's head to the neutral position and cleans the child's nostril. In step 118, the illustrative irrigation process proceeds on the child's other nostril, with the caregiver introducing hypertonic saline is introduced into the child's other nostril.

After introducing the saline into the child's nostril, in step 120 the caregiver blocks the nostril to prevent the hypertonic saline solution from escaping through the opening in the nostril. Then, in step 122, the caregiver tilts the child's head in the direction opposite the side of the nostril into which she has introduced the hypertonic saline. That is, if the solution has been introduced to the child's right nostril, the caregiver tilts the child's head to the left. The solution is allowed to travel, on an interior path, from the nostril into which the solution was introduced to the child's other nostril. In step 124, the caregiver returns the child's head to a neutral position (i.e., head tilted back, but tilted to neither the left or the right) and cleans the child's nostril, for example, using a suction bulb or syringe to suction fluid from the nostril that has had the hypertonic saline solution introduced.

The caregiver then, in step 126, again introduces hypertonic saline into and in step 128 blocks the child's nostril, as before. The caregiver then tilts the child's head in step 130 to the same side as the nostril into which they have just placed the hypertonic solution in order to irrigate the sinuses on that side of the child's head. In step 132 the caregiver again returns the child's head to the neutral position and cleans the child's nostril. The process is repeated as necessary, then ends in step 134. In an alternative embodiment, the child's head may be tilted backwards. A caregiver then introduces hypertonic saline into one or both nostrils and maintains the child's head in the tilted position long enough to allow gravity to drain the solution through the nares, down the posterior oropharyncx and on its way through the esophagus and into the stomach.

In another aspect of the invention, a sterile package of hypertonic saline contains a sufficient volume of hypertonic saline for a single use. In an illustrative embodiment, the saline is greater than 0.9% and less than 3.0% sodium chloride and each package contains between approximately 1 ml and 60 ml. Single-use packages in accordance with the principles of the present invention ensure that the contents remain sterile until use and provide for convenient delivery of the appropriate amount of solution. Such packages may take any of many forms, including various configurations of ampule, syringe, or squeeze bottle, for example. A syringe or squeeze bottle provides the advantage of allowing the operator to apply a controlled volume of the solution at a controlled rate. Additionally, the package may be made of any of a variety of materials, such as glass, plastic or rubber, for example. Ampules are known and discussed, for example, in U.S. Pat. No. 6,846,469, issued to Snedden, entitled, RESILIENT DISPERSING AMPULE AND PROCESS FOR FORMATION THEREOF, in U.S. Pat. No. 3,917,120, issued to Larenz et al, entitled, SINGLE USE CONTAINER FOR LIQUID PHARMACEUTICAL COMPOSITIONS, and in U.S. Pat. No. 5,320,257, issued to Snedden, entitled, RESILIENT AMPULE WITH ARTICULATING LINKAGE AND ELONGATE SPOUT, all of which are hereby incorporated by reference in their entirety. A wide variety of other single-use containers, such as syringes and squeeze bottles are also known.

The schematic diagram of FIG. 2 illustrates one form of ampule 200 that may be employed, in accordance with the principles of the present invention, as a single-use container for hypertonic saline solution. The ampule 200 includes neck 202 and reservoir 204 portions. Access to hypertonic saline solution contained within the reservoir 204 portion may be obtained by cutting or breaking the neck 202 portion off from the reservoir 204 portion. Some ampules provide a frangible area between the neck 202 and reservoir 204 portions to allow for relatively easy separation of the portions and access to the fluids contained within the reservoir.

The schematic diagram of FIG. 3 illustrates another type of ampule 300 that may be employed in accordance with the principles of the present invention. The ampule 300 includes neck 302 and reservoir 304 portions and a top 306 portion designed to twist off, thereby providing access to the contents of the reservoir 304.

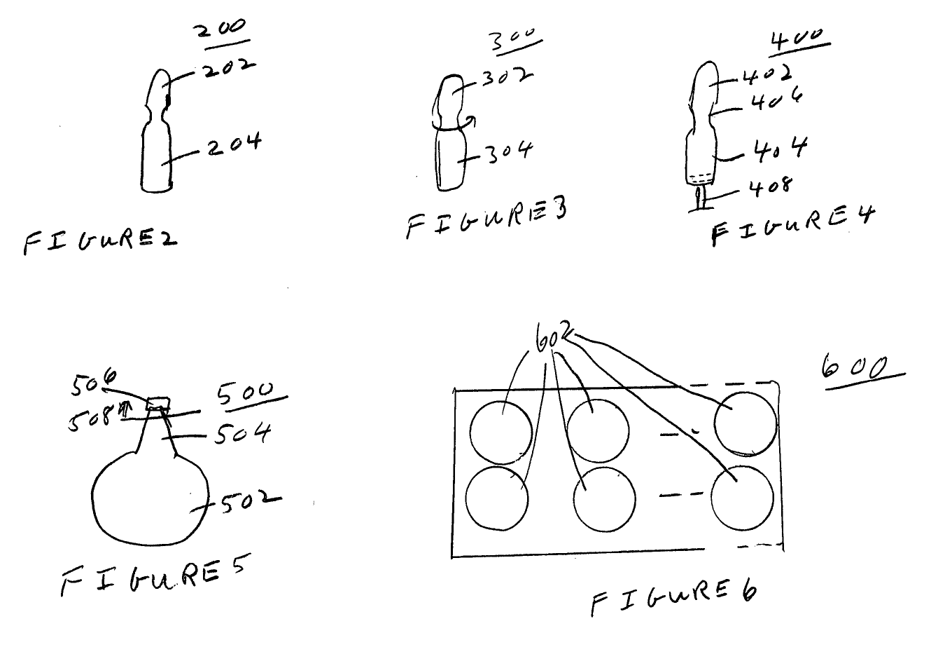

Another type of ampule suitable for single-use delivery of hypertonic saline solution in accordance with the principles of the present invention is illustrated in the schematic diagram of FIG. 4. The ampule 400 includes cap 402 and reservoir 404 portions and a frangible area 406 separating the two. In this illustrative embodiment a plunger mechanism 408 is included to allow the ampule 400 to act as a syringe, once opened.

A bulb dispenser 500 suitable for single-use delivery of hypertonic saline solution in accordance with the principles of the present invention is illustrated in the schematic diagram of FIG. 5. The bulb 502 form a reservoir for holding hypertonic saline solution. The neck 504 may be inserted into a nostril, of an infant, for example, for irrigation of the infant's sinuses in accordance with the principles of the present invention. The bulb dispenser 500 may include a cap 506 that is removable for use, or the neck 504 may be sealed, requiring a user to sever a top portion 508 of the neck 504 in order to dispense the hypertonic saline solution contained within the dispenser 500.

Any of the forgoing ampules may include a plunger mechanism that permits the ampule to operate as a syringe once the ampule is opened, by breaking a frangible area, or twisting off a cap, for example. Syringes and their operation are known.

The conceptual block diagram of FIG. 6 illustrates a kit 600 of single-use hypternonic saline solution packages 602, in accordance with the principles of the present invention. Each of the packages 602 contains sufficient hypertonic saline for a single application. The concentration of hypertonic solution may differ from single-use package to single-use package within the kit 600 and the entire kit 600 may contain a sufficient number of packages 602 for a full course of treatment. That is, for example, a kit 600 may contain seven packages 602 of hypertonic saline, one per day for a one-week course of treatment, with the concentration of hypertonic saline decreasing from the concentration of the saline within the package to be used on the first day of treatment. Longer-term treatment is contemplated in accordance with the principles of the present invention. The kit 600 may, for example, include sufficient packages for a five week course of treatment, for example. Additionally, the containers 602 may include a threaded lid.

The foregoing description of specific embodiments of the invention has been presented for the purposes of illustration and description. It is not intended to be exhaustive or to limit the invention to the precise forms disclosed, and many modifications and variations are possible in light of the above teachings. The embodiments were chosen and described to best explain the principles of the invention and its practical application, and to thereby enable others skilled in the art to best utilize the invention. It is intended that the scope of the invention be limited only by the claims appended hereto.

What is claimed is:

1. An apparatus comprising:
a single-use container; and
hypertonic saline solution within said single-use container.

2. The apparatus of claim 1 wherein the concentration of the saline falls within a range from 0.9% to 3.0% Sodium Chloride.

3. The apparatus of claim 1 wherein the volume of the container ranges between 1 ml and 60 ml.

4. The apparatus of claim 1 wherein the container is an ampule.

5. The apparatus of claim 1 wherein the container is a flexible bottle.

6. The apparatus of claim 2 wherein the container is a syringe.

7. The apparatus of claim 4 wherein the ampule is a twist-top plastic ampule.

8. An apparatus comprising:
a package;
a plurality of single-use containers contained within said package; and
hypertonic saline solution within each of said single-use containers.

9. The apparatus of claim 8 wherein each container within said package contains saline having the same concentration as every other container within the package.

10. The apparatus of claim 9 wherein the concentration of the saline within each of the containers falls within a range from 0.9% to 3.0% Sodium Chloride

11. The apparatus of claim 8 wherein containers within said package contain saline having concentrations that differ among the containers, and the concentration within each container falls within a range from 0.9% to 3.0% Sodium Chloride.

12. The apparatus of claim 8 wherein each container is an ampule.

13. The apparatus of claim 8 wherein each container is a flexible bottle.

14. The apparatus of claim 12 wherein the ampule are glass ampules.

15. The apparatus of claim 12 wherein the ampules are twist-top plastic ampules.

16. A method for treating juvenile sinus problems comprising the steps of:
tilting the juvenile's head back;
administering hypertonic saline solution to one or both nostrils of the juvenile; and
allowing the hyptertonic saline solution to travel by force of gravity through the juvenile's nasal passages.

17. The method of claim 16 wherein the saline solution is allowed to drain through the nares, down the posterior oropharynx and esophagus and into the juvenile's stomach.

18. The method of claim 16 further comprising the step of:
tilting the juvenile's head to allow the saline solution to drain from one nostril into the other.
